(19) United States
(12) Patent Application Publication
(10) Pub. No.: US 2025/0144027 A1
(43) Pub. Date: May 8, 2025

(54) METHOD FOR CARTILAGE REGENERATION AND REGROWTH FOLLOWING INJURY

(71) Applicant: ELUCIDERM INC., San Diego, CA (US)

(72) Inventors: Daniel Holsworth, San Diego, CA (US); Sarika Saraswati, Nashville, TN (US)

(21) Appl. No.: 18/691,159

(22) PCT Filed: Sep. 13, 2022
(86) PCT No.: PCT/US2022/043391

§ 371 Date: Mar. 12, 2024

Related U.S. Application Data
(60) Provisional application No. 63/243,638, filed on Sep. 13, 2021.

(51) Int. Cl. A61K 9/1271; A61K 31/519; A61K 45/06; A61K 47/10; A61K 47/26; A61K 47/38; A61K 47/69; A61P 19/04

(52) U.S. Cl. CPC A61K 9/1271; A61K 31/519; A61K 45/06; A61K 47/10; A61K 47/26; A61K 47/38; A61K 47/6911; A61P 19/04

(57) ABSTRACT

A method for enhancing cartilage regeneration following its wounding is provided which includes identifying a mammal as experiencing an injury to its cartilage applying to it a composition including graphene oxide (GO) and hyaluronic acid (HA) that are covalently linked, XAV939, and water. The composition of the method may also include a surfactant, such as PEG. The composition can be topically administered to a subject to treat cartilage wounding of the subject.

### CROSS REFERENCE TO RELATED APPLICATIONS

The present application claims from the benefit of U.S. provisional patent application No. 63/243,638 filed Sep. 13, 2021, such application expressly incorporated by reference herein for all purposes.

### FIELD

The present disclosure described herein relates to compositions and methods for treatment of cartilage injury, and in particular, the enhanced cartilage regeneration following cartilage injury.

### BACKGROUND

All publications, patents and patent applications cited within this application are herein incorporated by reference in their entirety to the same extent as if the disclosure of each individual publication, patent application or patent was specifically and individually indicated to be incorporated by reference in its entirety.

Cartilage represents an important form of connective tissue in mammals, providing both structural and functional activities in the body. Cartilage is comprised of three broad categories; the most widespread form being hyaline cartilage generally found at the end of bones forming part of a joint, elastic cartilage such as that found in the ears or nose which provides structure on the body, and fibrocartilage found at the insertions of ligaments and tendons.

While repair of cartilage is observed during fetal development or in the very young, repair and regeneration of cartilage following wounding in mature mammals is almost entirely absent. While the prior art has recognized the role of growth factors and wound lesion edge integration in the repair process, there has been an unmet need for therapeutics improving cartilage regeneration and healing following injury.

The Wnt pathway has been shown to play a key role in dermal fibrosis and scarring. The Wnt pathway is an evolutionary conserved pathway that regulates crucial aspects of cell fate determination, cell polarity, cell migration, neural patterning, and organogenesis during embryonic development. This pathway is instrumental in ensuring proper tissue development in embryos and tissue maintenance in adults. Wnt signaling is involved at the beginning stages of skin development. Following gastrulation, embryonic cells of the ectoderm and the mesoderm differentiate to form the epidermis and dermis, respectively.

Although there are at least three distinct Wnt signaling pathways involved in the signal transduction process, the canonical (or β-catenin dependent) Wnt pathway is the most understood. β-Catenin is the key effector molecule resulting from the signaling of the canonical Wnt pathway, and its protein levels are regulated through a “destruction complex”. In the absence of a Wnt signal, the transcriptional activator β-catenin is actively degraded in the cell by the actions of a protein complex, designated the “destruction complex”. Within this complex, Axin-1 and 2 with adenomatous polypsis coli form a scaffold that facilitates β-catenin phosphorylation by casein-kinase 19a and glycogen synthase kinase 3B. Phosphorylated β-catenin is recognized and ubiquitinylated, resulting in its proteosomal degradation. Tankryase I and II (TK1 and TK2) are poly(ADP-ribose) polymerases (PARPs) that function to parsylate and destabilize Axin-1 and 2 proteins, thus destabilizing the β-catenin destruction complex. Once the destruction complex is destabilized, this allows β-catenin to be dephosphorylated, and subsequently stabilized and allowed to accumulate in the cytoplasm and enter the cell nucleus, where it interacts with members of the Tcf/Lef family. β-catenin converts the Tcf proteins into potent transcriptional activators by recruiting co-activator proteins, thus ensuring efficient activation of Wnt target genes. The Wnt pathway, once activated by the Wnt family of natural ligands, upregulates TNK1 and TNK2 to help destabilize the destruction complex. Studies have shown that TNK1 and TNK2 are critical regulators of canonical Wnt signaling.

XAV939 is a small molecule that selectively inhibits Wnt/β-catenin-mediated transcription through TNK 1 and TNK2 inhibition with an IC₅₀ of 11 nM/4 nM in cell-free assays, regulates axin levels, and does not affect CRE, F-κB, or TGF-β. Topical application of XAV939 in a mouse ear punch assay demonstrated that XAV939 significantly increased rate of wound closure with reduced fibrosis (scarring). However, XAV939 was dissolved in DMSO and used only as a “research tool” compound due to its very low aqueous solubility (<1 μg/mL). The problem with this approach is that use of DMSO for topical administration can give rise to undesired pharmacological activity or adverse reactions. A soluble form of XAV939 suitable for humans is required for practical and medical use.

A matrix component comprising graphene oxide (GO) and hyaluronic acid (HA) has been shown to be effective in both providing a supportive matrix for XAV939 and allowing the use of XAV939 as a therapeutic for wound healing in humans and animals See for example US20210000959, where XAV939 in a GO-HA matrix provides substantial improvement to the speed and quality of wound healing; specifically causing the tissues to limit wound healing that follows a fibrotic healing pathway that leads to scarring. Further, increased cartilage regeneration and healing following acute injury has been reported, for example by way of a 2 mm biopsy punch wound made in the center of the cartilaginous region of a C57Bl/CJ mouse, by way of administration of XAV939 dissolved in DMSO (Bastakoty, D. et al. 2015, 29 (12): 4881-4892).

While any regeneration of cartilage following administration of a therapeutic is beneficial given the limited natural regenerative capacity of cartilage in non-fetal mammals, the art is in there is a need of improved compositions for inducing, enhancing or increasing cartilage regeneration following acute injury.

### SUMMARY

The present disclosure provides for a method for stimulating regeneration of mammalian cartilage subject to an injury, comprising optionally identifying a mammal as experiencing an injury to cartilage; optionally identifying the location and extent of the cartilage subject to an injury; and contacting of said cartilage subject to an injury with an effective amount of a composition, the composition comprising a matrix component consisting of a graphene oxide (GO) and hyaluronic acid (HA) conjugate (GO-HA), wherein the GO and HA are covalently linked via a linker; 3,5,7,8-tetrahydro-2-[4-(trifluoromethyl)phenyl]-4H-thiopyrano[4,3-d]pyrimidin-4-one (XAV939); and water optionally wherein XAV939 constitutes from about 0.001 wt % to about 5 wt % of the total composition. In one embodiment said cartilage subject to an injury is cartilage subject to an acute injury. In a further embodiment said cartilage subject to injury is elastic cartilage. In another embodiment the composition further comprises a surfactant. In a further embodiment the surfactant is polyethylene glycol (PEG), or a poloxamer. In a still further embodiment the PEG has a molecular weight of from about 200 to about 400 Daltons. In a still further embodiment the PEG is in an amount of from about 0.1 wt % to about 20 wt % of the total composition. In another embodiment the composition further comprises a thickener. In a further embodiment the thickener comprises hydroxypropyl cellulose (HPC). In another embodiment the linker comprises 2-25 carbons. In a further embodiment the linker comprises one or more —CH₂CH₂O— units. In another embodiment the linker comprises —Rₓ—R_{S}—R_{y}—, wherein Rₓ and R_{y} are each independently selected from the group consisting of —CO—, —COO—, —NH—, —NH—NH—, —NH—NH—CO—, —CS—, —S—, and —O—, and wherein R_{S} is an unsubstituted, saturated or unsaturated, linear alkylene group having 2-20 backbone carbons. In a further embodiment Rₓ and R_{y} are each —NH—NH—CO—. In another embodiment the weight ratio of XAV939 to GO-HA is from about 1:2 to about 2:1. In another embodiment the GO-HA constitutes from about 0.001 wt % to about 5 wt % of the total composition.

In another aspect the present disclosure provides for a method for stimulating regeneration of mammalian cartilage subject to an injury, comprising optionally identifying a mammal as experiencing an injury to cartilage; optionally identifying the location and extent of the cartilage subject to an injury; and contacting of said cartilage subject to an injury with an effective amount of a composition, once every 48 hours, the composition comprising a matrix component comprising a graphene oxide (GO) and hyaluronic acid (HA) conjugate (GO-HA), wherein the GO and HA are covalently linked via a linker; 3,5,7,8-tetrahydro-2-[4-(trifluoromethyl)phenyl]-4H-thiopyrano[4,3-d]pyrimidin-4-one (XAV939); and water optionally wherein XAV939 constitutes from about 0.001 wt % to about 5 wt % of the total composition. In one embodiment said cartilage subject to an injury is cartilage subject to an acute injury. In a further embodiment said cartilage subject to injury is elastic cartilage. In another embodiment the composition further comprises a surfactant. In a further embodiment the surfactant is polyethylene glycol (PEG), or a poloxamer. In a still further embodiment the PEG has a molecular weight of from about 200 to about 400 Daltons. In a still further embodiment the PEG is in an amount of from about 0.1 wt % to about 20 wt % of the total composition. In another embodiment the composition further comprises a thickener. In a further embodiment the thickener comprises hydroxypropyl cellulose (HPC). In another embodiment the linker comprises 2-25 carbons. In a further embodiment the linker comprises one or more —CH₂CH₂O— units. In another embodiment the linker comprises —Rₓ—R_{S}—R_{y}—, wherein Rₓ and R_{y} are each independently selected from the group consisting of —CO—, —COO—, —NH—, —NH—NH—, —NH—NH—CO—, —CS—, —S—, and —O—, and wherein R_{S} is an unsubstituted, saturated or unsaturated, linear alkylene group having 2-20 backbone carbons. In a further embodiment Rₓ and R_{y} are each —NH—NH—CO—. In another embodiment the weight ratio of XAV939 to GO-HA is from about 1:2 to about 2:1. In another embodiment the GO-HA constitutes from about 0.001 wt % to about 5 wt % of the total composition.

In another aspect the present disclosure provides for a composition for use in a method for stimulating regeneration of mammalian cartilage subject to an injury the composition comprising a matrix component comprising a graphene oxide (GO) and hyaluronic acid (HA) conjugate (GO-HA), wherein the GO and HA are covalently linked via a linker; 3,5,7,8-tetrahydro-2-[4-(trifluoromethyl)phenyl]-4H-thiopyrano[4,3-d]pyrimidin-4-one (XAV939); and water; optionally wherein XAV939 constitutes from about 0.001 wt % to about 5 wt % of the total composition; and wherein the method optionally comprises identifying a mammal as experiencing an injury to cartilage; optionally identifying the location and extent of the cartilage subject to an injury; and contacting of said cartilage subject to an injury with an effective amount of said composition. In one embodiment said cartilage subject to an injury is cartilage subject to an acute injury. In a further embodiment said cartilage subject to injury is elastic cartilage. In another embodiment the composition further comprises a surfactant. In a further embodiment the surfactant is polyethylene glycol (PEG), or a poloxamer. In a still further embodiment the PEG has a molecular weight of from about 200 to about 400 Daltons. In a still further embodiment the PEG is in an amount of from about 0.1 wt % to about 20 wt % of the total composition. In another embodiment the composition further comprises a thickener. In a further embodiment the thickener comprises hydroxypropyl cellulose (HPC). In another embodiment the linker comprises 2-25 carbons. In a further embodiment the linker comprises one or more —CH₂CH₂O— units. In another embodiment the linker comprises —Rₓ—R_{S}—R_{y}—, wherein Rₓ and R_{y} are each independently selected from the group consisting of —CO—, —COO—, —NH—, —NH—NH—, —NH—NH—CO—, —CS—, —S—, and —O—, and wherein R_{S} is an unsubstituted, saturated or unsaturated, linear alkylene group having 2-20 backbone carbons. In a further embodiment Rₓ and R_{y} are each —NH—NH—CO—. In another embodiment the weight ratio of XAV939 to GO-HA is from about 1:2 to about 2:1. In another embodiment the GO-HA constitutes from about 0.001 wt % to about 5 wt % of the total composition.

In another aspect the present disclosure provides for a composition for use in a method for stimulating regeneration of mammalian cartilage subject to an injury the composition comprising a matrix component comprising a graphene oxide (GO) and hyaluronic acid (HA) conjugate (GO-HA), wherein the GO and HA are covalently linked via a linker; 3,5,7,8-tetrahydro-2-[4-(trifluoromethyl)phenyl]-4H-thiopyrano[4,3-d]pyrimidin-4-one (XAV939); and water; wherein XAV939 constitutes from about 0.001 wt % to about 5 wt % of the total composition; and wherein the method comprises identifying a mammal as experiencing an injury to cartilage; identifying the location and extent of the cartilage subject to an injury; and contacting of said cartilage subject to an injury with an effective amount of said composition, once every 48 hours. In one embodiment said cartilage subject to an injury is cartilage subject to an acute injury. In a further embodiment said cartilage subject to injury is elastic cartilage. In another embodiment the composition further comprises a surfactant. In a further embodiment the surfactant is polyethylene glycol (PEG), or a poloxamer. In a still further embodiment the PEG has a molecular weight of from about 200 to about 400 Daltons. In a still further embodiment the PEG is in an amount of from about 0.1 wt % to about 20 wt % of the total composition. In another embodiment the composition further comprises a thickener. In a further embodiment the thickener comprises hydroxypropyl cellulose (HPC). In another embodiment the linker comprises 2-25 carbons. In a further embodiment the linker comprises one or more-CH₂CH₂O— units. In another embodiment the linker comprises —Rₓ—R_{S}—R_{y}—, wherein Rₓ and R_{y} are each independently selected from the group consisting of —CO—, —COO—, —NH—, —NH—NH—, —NH—NH—CO—, —CS—, —S—, and —O—, and wherein R_{S} is an unsubstituted, saturated or unsaturated, linear alkylene group having 2-20 backbone carbons. In a further embodiment Rₓ and R_{y} are each —NH—NH—CO—. In another embodiment the weight ratio of XAV939 to GO-HA is from about 1:2 to about 2:1. In another embodiment the GO-HA constitutes from about 0.001 wt % to about 5 wt % of the total composition.

In some or any of the foregoing embodiments, XAV939 constitutes from about 0.001 wt % to about 5 wt % of the total of XAV939, GO-HA, and water.

In some or any of the foregoing embodiments, GO-HA constitutes from about 0.001 wt % to about 5 wt % of the total of XAV939, GO-HA, and water.

### BRIEF DESCRIPTION OF THE FIGURES

FIG. 1 shows the distance between opposing cartilage endpoints in mice as between 30-day treatment of biopsy punch wounds with saline and XAV939 in DMSO and 15-day treatment with GO-HA, and ELU42;

FIG. 2 shows the distance between opposing cartilage endpoints in mice as between 30-day treatment of biopsy punch wounds with XAV939 in DMSO and 15-day treatment with ELU42;

FIG. 3 shows the distance between opposing cartilage endpoints in mice as between 15-day treatment with GO-HA and 15 ELU42; and

FIG. 4 shows the distance between opposing cartilage endpoints in rabbits as between 21-day treatment of biopsy punch wounds with saline and 21-day treatment with ELU42,

### DETAILED DESCRIPTION

The present disclosure provides for novel methods of administration of compositions of the present disclosure to induce improved cartilage regeneration and healing.

It is to be understood that the description of compounds, compositions, formulations, and methods of treatment described herein include “comprising,” “consisting of,” and “consisting essentially of” embodiments. In some embodiments, for all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the basic and novel properties (in some embodiments, the condition being treated), but do not contain any other components which substantially affect the basic and novel properties (in some embodiments, condition being treated) other than those components expressly listed; or, if the composition does contain extra components other than those listed which substantially affect the basic and novel properties (in some embodiments, the condition being treated), the composition does not contain a sufficient concentration or amount of the extra components to substantially affect the basic and novel properties (in some embodiments, the condition being treated). When a method is described as “consisting essentially of” the listed steps, the method contains the steps listed, and may contain other steps that do not substantially affect the basic and novel properties (in some embodiments, the condition being treated), but the method does not contain any other steps which substantially affect the basic and novel properties (in some embodiments, the condition being treated) other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the basic and novel properties (in some embodiments, the condition being treated).

As used herein “alkyl” refers to straight or branched hydrocarbon. An alkyl may be linear, branched, cyclic, or a combination thereof, and may contain, for example, from one to sixty carbon atoms. Examples of alkyl groups include but are not limited to ethyl, ethyl, propyl, isopropyl, cyclopropyl, butyl isomers (e.g. n-butyl, iso-butyl, tert-butyl, etc.) cyclobutyl isomers (e.g. cyclobutyl, methylcyclopropyl, etc.), pentyl isomers, cyclopentane isomers, hexyl isomers, cyclohexane isomers, and the like.

As used herein, the term “linear alkyl” refers to a chain of carbon and hydrogen atoms (e.g., ethane, propane, butane, pentane, hexane, etc.).

As used herein, the term “branched alkyl” refers to a chain of carbon and hydrogen atoms, without double or triple bonds that contains a fork, branch, and/or split in the chain. “Branching” refers to the divergence of a carbon chain, whereas “substitution” refers to the presence of non-carbon/non-hydrogen atoms in a moiety.

As used herein, the term “cycloalkyl” refers to a completely saturated mono- or multi-cyclic hydrocarbon ring system. When composed of two or more rings, the rings may be joined together in a fused, bridged or spiro-connected fashion. A cycloalkyl group may be unsubstituted, substituted, branched, and/or unbranched. Typical cycloalkyl groups include, but are not limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated. Unless specified otherwise (e.g., substituted cycloalkyl group, heterocyclyl, cycloalkoxy group, halocycloalkyl, cycloalkylamine, thiocycloalkyl, etc.), an alkyl group contains carbon and hydrogen atoms only.

As used herein, the term “heteroalkyl” refers to an alkyl group, wherein one or more carbon atoms are independently replaced by one or more heteroatoms (e.g., oxygen, sulfur, nitrogen, phosphorus, silicon, or combinations thereof). The alkyl group containing the non-carbon substitution(s) may be a linear alkyl, branched alkyl, cycloalkyl (e.g., cycloheteroalkyl), or combinations thereof. Non-carbons may be at terminal locations (e.g., 2-hexanol) or integral to an alkyl group (e.g., diethyl ether).

As used herein “alkoxy”, used alone or in combination, means the group —O— alkyl.

As used herein “alkenyl”, used alone or in combination, means a straight or branched chain hydrocarbon having at least 2 carbon atoms, which contains at least one carbon-carbon double bond.

As used herein “alkynyl”, used alone or in combination, means a straight or branched chain hydrocarbon having at least 2 carbon atoms, which contains at least one carbon-carbon triple bond.

As used herein “amine” or “amino” as used herein are represented by a formula NA₁A₂A₃, where A₁, A₂, and A₃ can be, independently, hydrogen or optionally substituted alkyl, cycloalkyl, alkenyl, cycloalkenyl, alkynyl, cycloalkynyl, aryl, or heteroaryl group as described herein. In specific embodiments amine refers to any of NH₂, NH(alkyl), NH(aryl), N(alkyl)₂, N(alkyl)(aryl), and N(aryl)₂.

As used herein, “acute injury” means damage or wounds caused by, among others, traumatic force, chemical toxicity, thermal burns, frostbite, acute ischemia, and reperfusion injury. Exemplary traumatic force injury includes, among others, surgical procedures and blunt force trauma (e.g., gunshot wounds, knife wounds, etc.). The compositions may be applied on or injected into the affected tissues to promote vascularization, repair, and regeneration of such damaged tissues.

As used herein “regeneration” means the growth of destroyed or devitalized tissue from the remnant tissue. It is a reparative attempt of the body, and in the context of cartilage represents the migration or replication of chondrocytes, or transformation of progenitor cells into chondrocytes; giving rise to an organization of said chondrocytes to form hyaline, elastic or fibrocartilage.

As used herein a “thickener” is a substance which increases the viscosity of a liquid without substantial modification to the chemical or biological properties of the liquid.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index (Merck & Company, Rahway, N.J.), considerations for the inclusion of various components in pharmaceutical compositions are described, (e.g., Gilman et al. (Eds.), 2010, Goodman and Gilman's: The Pharmacological Basis of Therapeutics, 12th Ed., The McGraw-Hill Companies).

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297.

A “therapeutically effective amount” or “pharmaceutically effective amount” of a compound as provided herein, is one which is sufficient to achieve the desired effect and may vary according to the nature and severity of the disease condition, and the potency of the compound. “Therapeutically effective amount” is also intended to include one or more of the compositions of the present disclosure so as to result in the increased regeneration of cartilage following an acute injury. The combination of compounds and/or compositions is preferably a synergistic combination. Synergy, as described in the art (for example, Chou, 2010, Canc. Res. 70(2):440-446), occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age and medical history.

The term “mammal” is used in its usual biological sense. Thus, it specifically includes humans, cattle, horses, dogs, and cats, but also includes many other species.

In one aspect of the disclosure, a composition for treating cartilage subject to an acute injury is provided, which includes: a matrix component comprising a conjugate of graphene oxide (GO) and hyaluronic acid (HA) where GO and HA are covalently linked via a linker; XAV939; and water. The covalently-linked GO and HA is also referred to herein as GO-HA conjugate or simply GO-HA.

XAV939 is a potent tankyrase inhibitor, with a chemical name 3,5,7,8-Tetrahydro-2-[4-(trifluoromethyl)phenyl]-4H-thiopyrano[4,3-d]pyrimidin-4-one. The structure of XAV939 is shown below:

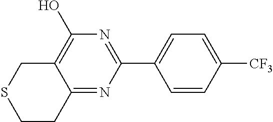 [SMILES: Oc1nc(-c2ccc(C(F)(F)F)cc2)nc2c1CSCC2]

Graphene oxide (GO) as used herein refers to an oxidized form of graphene, which is a single layer form of graphite. GO can be obtained by treating graphite with strong oxidizers. GO contains carbon, oxygen, and hydrogen in various amounts, depending on how it is made. It can have several hundreds of nanometers, up to several micrometers, its planar direction, and about 0.7-1.2 nm in thickness. GO can include various oxygen containing moieties, such as oxygen epoxide groups, carboxylic acid (—COOH), phenol, etc., when prepared using sulfuric acid (e.g. Hummers method). An example GO structure is shown below.

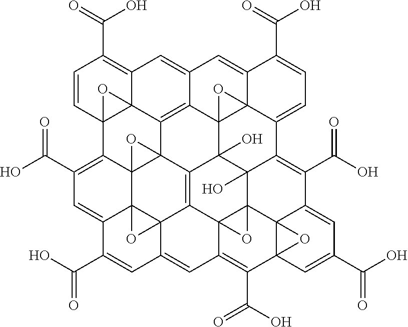

Hyaluronic acid (HA) is an anionic, highly hydrophilic, non-sulfated glycosaminoglycan, occurring naturally throughout the human body. It can be several thousands of carbohydrate units long and can bind to water giving it a gel of stiff viscous quality. An example structure of HA is provided below:

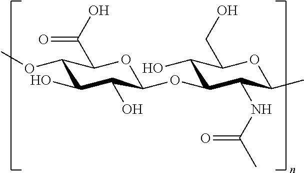

In a composition of present disclosure, the GO and HA are covalently linked to form a matrix component (or a carrier), which can serve to solubilize XAV939 as well as providing other simultaneous benefits to wound healing. The covalent linking can be accomplished by using a linker (or linker moiety). In some embodiments, the linker can include 2-25 carbons. In some embodiments, the linker is linear. In other embodiments, the linker is branched. The linker can be saturated or unsaturated.

In some embodiments, the linker can comprise a C₂-C₂₅ alkylene group, where the carbons and hydrogens in the alkylene group can be substituted by oxygen or other atoms or groups such as hydroxy, carboxy, amino, alkyl, alkoxy, alkenyl, alkynyl, nitro, etc. In some embodiments, the linker can comprise one or more —CH₂CH₂O— units.

In some embodiments, the linker comprises —Rₓ—R_{S}—R_{y}—, wherein Rₓ and R_{y} are each independently selected from the group consisting of —CO—, —COO—, —H—, —H—H—, —H—H—CO—, —CS—, —S—, -0-, and wherein Rₛ (which is also referred to as the spacer group in this application) can be an unsubstituted or substituted, saturated or unsaturated linear alkylene group having 2-20 backbone carbons. In particular embodiments, both Rₓ and R_{y} are *—H—H—CO— (* denoting the ends of the linker distal to Rs).

In some embodiments of the composition, the weight ratio of XAV939 to GO-HA can be from about 1:100 to 100:1, e.g., from about 1:2 to about 2:1. In some embodiments, in the GO-HA conjugate, the weight ratio of GO:HA can be from about 1:1 to about 1:20, or from about 1:6 to about 1:10.

Other non-ionic hydrophilic material such as copolymers of PEG and PPG (polypropylene glycol), e.g., poloxamers, can also be used. In one example, Poloxamer-188 (which has an average molecular weight of about 8400 Daltons) can be used.

In some embodiments, the composition further comprises pharmaceutical carriers, excipients, compound(s) in addition to XAV939, or materials which enable the compositions to be presented in topically administrable semi-solid aqueous gel forms. For example, carboxymethylcellulose can be used as a gel-forming agent. However, other cellulose derivatives such as microcrystalline cellulose as well as polysaccharides such as alginate, agarose, tragacanth, guar gum, and xanthum gum; are also suitable as gel-forming agents. The gel may, if required, be made thicker and/or stiffer by addition of a relatively resilient gel-forming material such as a cross-linked fibrous protein, e.g. gelatin or collagen cross-linked with formaldehyde. In some embodiments, the composition can be in a form of a cream, which can include those excipients suitable for a cream formulation, such as paraffin oil, vaseline, wax, organic esters such as cetyl palmitate, etc.

In some embodiments, the composition of the present disclosure further comprises a thickener for desired viscosity of the composition for skin delivery. For example, the thickener can include hydroxypropyl cellulose (HPC), or xanthan gum. HPC can make the composition into a smooth film for easy application. It also reduces evaporation and allows the wound to stay moist longer, a factor that has been shown to improve healing and result in decreased scarring. There are different grades of HPC available according to molecular weights or viscosity of certain concentrations of HPC water solution.

In some embodiments of the composition, XAV939 can constitute from about 0.001 wt % to about 5 wt % of the total composition (including water). In some embodiments, XAV939 can constitute from about 0.01 wt % to about 2 wt %, from about 0.02 wt % to about 1 wt %, or from about 0.05 wt % to about 0.5 wt % of the total composition. In some embodiments, GO-HA constitutes from about 0.001 wt % to about 5 wt % of the total composition. In some embodiments, GO-HA can constitute from about 0.01 wt % to about 2 wt %, from about 0.02 wt % to about 1 wt %, or from about 0.05 wt % to about 0.5 wt % of the total composition. In some or any of the foregoing embodiments, the wt % of XAV939 is based on the total of XAV939, GO-HA, and water. In some or any of the foregoing embodiments, the wt % of GO-HA is based on the total of XAV939, GO-HA, and water.

XAV939 is evenly dispersed in the viscous suspension, which is stable at room temperature for months. In some embodiments, the composition further comprises a surfactant that enhances mixability or solubility of hydrophobic substances in water. In some examples, the surfactant can be a non-ionic hydrophilic material such as polyethylene glycol (PEG). The PEG can have a number-averaged molecular weight of from about 100 to about 10,000 Daltons, or about 200 to about 4000 Daltons, e.g., from about 200 to about 1000, from about 200 to about 800, from about 200 to about 500, from about 200 to about 400, from about 300 to about 400, from about 350 to about 450, about 200, about 250, about 300, about 350, about 400, about 450, about 500, about 550, about 600, about 650, about 700, about 750, about 800, about 850, about 900, about 950, about 1000 Daltons, etc. In some embodiments, the PEG can be present in the composition in an amount of from about 0.1 to about 20 wt % of that of the total composition. For example, the PEG can be from about 0.2 wt % to about 10 wt %, or from about 0.5 wt % to about 10 wt %, or from about 1 wt % to about 10 wt % of the total composition.

Provided is a composition where the composition is a liposomal formulation. Provided is a composition where the composition comprises a matrix component comprising a graphene oxide (GO) and hyaluronic acid (HA) conjugate (GO-HA), wherein the GO and HA are covalently linked via a linker; XAV939; water; and butylene glycol. In some or any embodiments, the composition comprises a matrix component comprising a graphene oxide (GO) and hyaluronic acid (HA) conjugate (GO-HA), wherein the GO and HA are covalently linked via a linker; XAV939; water; and a polysorbate 20. In some or any embodiments, the composition comprises a matrix component comprising a graphene oxide (GO) and hyaluronic acid (HA) conjugate (GO-HA), wherein the GO and HA are covalently linked via a linker; XAV939; water; and phosphatidylcholine. In some or any embodiments, the composition comprises a matrix component comprising a graphene oxide (GO) and hyaluronic acid (HA) conjugate (GO-HA), wherein the GO and HA are covalently linked via a linker; XAV939; water; and ethanol. In some or any embodiments, the composition comprises a matrix component comprising a graphene oxide (GO) and hyaluronic acid (HA) conjugate (GO-HA), wherein the GO and HA are covalently linked via a linker; XAV939; water; and hydroxypropylcellulose. Any of the foregoing embodiments in this paragraph can be combined with each other; in particular, the liposomal formulation comprises XAV939, butylene glycol, a polysorbate 20, phosphatidylcholine, ethanol, GO-HA, water, and hydroxypropylcellulose. In some or any embodiments, the liposomal formulation is used to practice any of the embodiments as described herein, including those in the claims.

In the compositions as described herein, other pharmaceutical or therapeutic compounds may be included in addition to XAV939. In other words, the compositions with XAV939 present can also serve as a base dispersion medium in which other pharmaceutical or therapeutic agents, especially those which are hydrophobic, may be dispersed, e.g., for topical administration to cartilage subject to acute injury. These agents may include antifibrotic compounds such as pirfenidone, halofuginone, nintedanib, tocilizumab, rilonacept, etc., anti-cancer agents, anti-inflammatory agents, analgesics, antibiotics, etc.

In one aspect, the present disclosure provides for a method of stimulating regeneration of cartilage subject to an acute injury, the method comprising contacting the cartilage with an effective amount of the composition as described herein. The cartilage subject to an acute injury is contemplated to include, but not be limited to, those that arise from a surgical wounding caused by a physical impact that disrupts the structure and function of the cartilage (such as a laceration, abrasion, cut, scratch or puncture by a knife, scalpel, or other sharp or blunt objects) or that arise from a physical impact in a non-surgical setting that disrupts the structure and function of the cartilage (such as by a knife, bullet, or other sharp or blunt objects). The present disclosure contemplates cartilage subject to an acute injury by way of excessive (low or high) temperature such as a burn, ionizing radiation, chemotherapy, or unplanned acute injuries arising from accident or misadventure.

In some embodiments, the method of treatment can include delivering a second medication or therapeutic agent to the cartilage subject to an acute injury, comprising one or more of: corticosteroid, a cytotoxic drug, an antibiotic, an antiseptic, nicotine, an anti-platelet drug, an NSAID, colchicine, an anti-coagulant, a vasoconstricting drug or an immunosuppressive, a growth factor, an antibody, a protease, a protease inhibitor, an antibacterial peptide, an adhesive peptide, a hemostatic agent, living cells, honey, or nitric oxide. These therapeutic agents can be delivered as separate dosage forms from the compositions described herein, or may be included as additional components of the compositions described herein, hence delivered together with XAV939.

The composition(s) of the present disclosure described herein can be administered by applying the composition(s) topically on the cartilage subject to an acute injury. If the composition is included in a medical device described herein which includes a substrate such as a patch or a pad, the medical device can be secured to the acute injury site such that the composition contacts the cartilage.

In the preparation method of compositions used in the methods of the present disclosure, the spacer group can be an unsubstituted or substituted, saturated or unsaturated linear alkylene group having 2-20 backbone carbons. For illustration and not limitation, the reagent for derivatizing HA can be selected from the following:

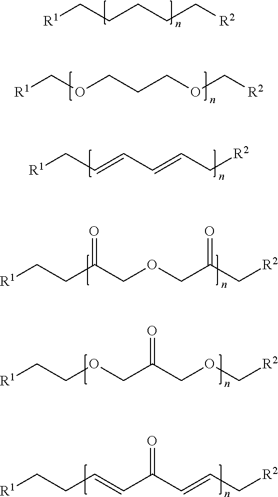

where R¹ and R² can be independently —CO HNH₂, —SH, —H₂, —OH, or other nucleophiles, and n is an integer and can be for example, 1-20, e.g., 1, 2, 3, 4, 5, 6, 7, 8, 9, 10, etc. In some embodiments, the reagent for derivatizing HA can be a dihydrazide, such as adipic acid dihydrazide.

In some embodiments, a method for preparing a composition of the present disclosure includes obtaining GO-HA (e.g., by the methods above), dissolving the GO-HA conjugate in water to obtain a GO-HA water solution, and adding XAV939 to the GO-HA water solution to form a mixture. In some examples, this is accomplished by dissolving XAV939 first in a non-ionic hydrophilic polymer, e.g., PEG-400 (or PEG 400, having an average molar mass of about 400), and then the XAV939 solution is added into the GO-HA conjugate water solution.

The present disclosure arises from the novel and unexpected finding of significantly increased regeneration of cartilage following acute injury in mammals, both in the quantity of regrowth as well as the quality of the cartilage regeneration and regrowth. While increased regeneration of cartilage following administration of Wnt inhibitors such as XAV939 is known in the art, as well as the benefits of providing XAV939 in a matrix of GO-HA; the significant increase in cartilage regeneration of injured cartilage with the timely administration of XAV939 in a matrix comprising GO-HA is both novel and unexpected.

Decreased rates of administration of the compositions of the present disclosure, as compared to administration of the active XAV939 dissolved in DMSO, demonstrate increased cartilage regeneration following acute cartilage injury in animal models recognized by the art as providing reduced healing rates (see, for example, Sullivan S. R., 2004, Plast. Reconstr. Surg. 113:953-60), provided significantly improved quantity of cartilage regrowth and improved quality of cartilage generation; in half the time as compared to XAV939 in DMSO.

The following examples are provided for purpose of illustration of certain aspects of the description herein and should not be deemed to limit the invention in any way.

### Example 1: Preparation of Wound-Treating Composition

The procedure of preparing the composition is disclosed in U.S. Pat. No. 11,369,685 B2. In brief, graphene oxide (1.25 g, 250 mL of 5 mg/mL GO dispersion in deionized water; supplier: Goographene Inc.) was added to 250 mL of ultra-pure de-ionized water and stirred for 5 minutes. Sodium hydroxide pellets (3 g (0.075 moles; supplier: Sigma-Aldrich) were added in small solid portions to the mixture over 30 minutes. Once addition was complete, it was stirred for 1 hour at room temperature. Next the solution was ultrasonicated for 30 minutes, and then chloroacetic acid (3.54 g, 0.0375 moles; supplier: Alfa Aesar) was added in small, solid portions over 20 minutes. The reaction mixture was then stirred for 18 hours at room temperature. The reaction mixture was acidified with hydrochloric acid (7 mL, 12 N). The solution was then transferred to centrifuge tubes and centrifuged for 15 minutes at 5,000 rpm. The water layer was then decanted and more ultra-pure deionized water (˜30 mL) added to the tubes before re-centrifugation. This process was repeated 3 times. Methanol (˜30 mL) was added to the precipitated, modified graphene oxide remaining in the centrifuged tubes and centrifuged at 5,000 rpm for 15 minutes. This process was repeated 3 times. Once the methanol was decanted, the tubes were put under vacuum for 48 hours at room temperature for drying. The product was then derivatized by addition of 1-(3-dimethylaminopropyl)-3-ethyl carbodiimide hydrochloride (52 mg, 0.00027 moles, supplier: Alfa Aesar) while maintaining pH with 0.1 N hydrochloric acid, and then added dropwise to a separate mixture containing adipic dihydrazide (17 mg, 0.00027 moles; supplier: Alfa Aesar) in 5 mL of ultra-pure deionized water at room temperature. Once the addition was complete, it was stirred at room temperature for 18 hours. The solution was then subjected to dialysis (MWC=3500) for 24 hours and lyophilized, resulting in the GO-HA referenced further herein.

11 mg of the GO-HA the was dissolved in 11 mL of ultra-pure water, to create an effective concentration of 1 mg/mL. The solution was ultrasonicated for 10 minutes. XAV939 (11 mg; supplier: APEBIO) was added to PEG-400 (0.5 mL) and subjected to ultrasonication for 30 minutes. The XAV939 solution was added dropwise to the GO-HA and vigorously stirred for 5 minutes. The combined solution was then subjected to ultrasonication for 1 hour and then stirred at room temperature for 18 hours. Hydroxypropyl cellulose (0.2 g; supplier: Sigma-Aldrich) was added in small portions at room temperature with vigorous stirring. Once addition was complete, the solution was stirred at room temperature for 24 hours to form a viscous solution of the GO-HA/XAV939 complex. The complex was used with a final concentration of 1.0 mg/mL XAV939 in 1.0 mg/mL GO-HA, and 0.182 g/mL hydroxypropyl cellulose (ELU42).

### Example 2: Cartilage Regrowth in Mice Following Administration of a Compound of the Present Disclosure

C57Bl/6J mice were purchased from The Jackson Laboratory (Bar Harbor, ME, USA) and maintained by PPY. C57Bl/6J mice (at least 3 months of age) were anesthetized with 3-5% isoflurane in O₂ administered using Tabletop Laboratory Animal Anesthesia System (VetEquip Inc., Pleasanton, CA, USA). A 2 mm biopsy punch wound was made in the center of the cartilaginous region of each ear using a disposable biopsy punch (Acuderm Inc., Fort Lauderdale, FL, USA) as described previously (Rai, M. F. et al, 2021, Arthritis Rheum., 64:2300-2310). The ears were treated topically with 5 μL/ear of XAV939 (Selleck Chemicals, S1180, Houston, TX, USA), dissolved to 5 μM in dimethyl sulfoxide (DMSO) or a control solution of DMSO, every day, for 30 days, resulting in a daily administration of 7.81 μg XAV939.

After 30 days of treatment, the ears were imaged using a Nikon Coolpix 8700 digital camera (Nikon Corporation, Japan). In order to quantify closure of the wounds, ears were excised following mouse sacrifice, fixed and placed on microscope slides for examination by way of the digital camera being affixed to a Micromaster Inverted microscope (Thermo Fisher Scientific, Waltham, MA, USA). Fixation was by way of immersion in 10% buffered formalin for twenty-four hours, sliced longitudinally across the injury and then embedded in paraffin for sectioning. Sections of the tissue were then stained with Trichome blues, imaged and analyzed by way of an Olympus DP71 microscope camera (Olympus America, Center Valley, PA, USA).

For ELU42 administration, STZ-induced type I diabetic C57Bl/6J mice were used. 8 to 12-week-old, diabetes induced mice, with serum glucose levels between 270-478 mg/dL were initiated into the study. The Mice were anesthetized with 3-5% isoflurane in O₂ administered using Tabletop Laboratory Animal Anesthesia System (VetEquip Inc., Pleasanton, CA, USA). A 2 mm biopsy punch wound was made in the center of the cartilaginous region of each ear using a disposable biopsy punch (AcudermInc., Fort Lauderdale, FL, USA) as described previously (Rai, M. F. et al, 2021, Arthritis Rheum., 64:2300-2310). The ears were treated topically with 50 μL of ELU42, prepared as described in Example 1, containing 1 mg/mL XAV939 (n=6) or a control solution of GO-HA, every other day for 15 days. For quantification of wound closure, ears were excised after the mice were sacrificed, placed on microscope slides (Denville Scientific, South Plainfield, NJ, USA) and imaged using Nikon Coolpix 8700 affixed to Micromaster Inverted microscope (Thermo Fisher Scientific, Waltham, MA, USA). Excised ears were fixed for 24 h in 10% buffered formalin, cut longitudinally across the injury and embedded in paraffin blocks. Sections of the embedded tissue were stained with Trichrome blue imaged and analysed using an Olympus DP71 microscope camera (Olympus America, Center Valley, PA, USA).

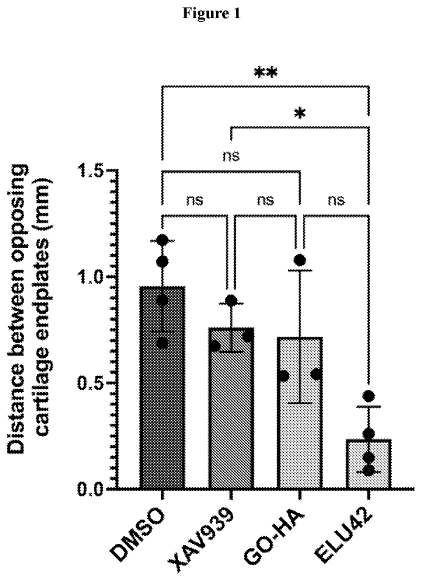

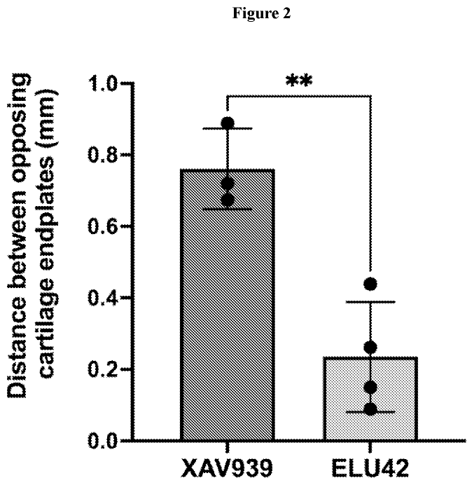

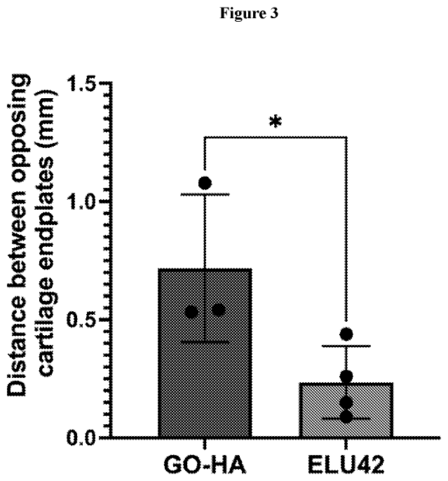

The distance between opposing cartilage endplates was measured in four separate animal groups following treatment of the biopsy punch wounds: XAV939 in DMSO every day for 30 days, DMSO (as a control) every day for 30 days, ELU42 every other day for 15 days, and GO-HA (as a control) every other day for 15 days. FIG. 1 shows the distance between opposing cartilage endpoints, as between 30-day treatment of biopsy punch wounds with XAV939 in DMSO, 15 day treatment of biopsy punch wounds with GO-HA, 30 day treatment of biopsy punch wounds with saline, and 15-day treatment with ELU42; where administration of ELU42 provided significant (ANOVA *p<0.05, unpaired t-test **p<0.001) quantitative improvement in cartilage regrowth with lesser dose of the active ingredient XAV939 administered for half the time as compared, with significant difference observed between ELU42 and DMSO (ANOVA **p<0.0001). There was significantly significant improvement observed for ELU42 compared to GO-HA (unpaired t-test *p<0.05). Further, the nature and character of the cartilage regrowth in the wounds was observed as improved in the XAV939 in DMSO and ELU42, as compared to DMSO and GO-HA controls, respectively. FIG. 2 shows a direct comparison of the distance between opposing cartilage endpoints, as between 30-day treatment of biopsy punch wounds with XAV939 in DMSO and 15-day treatment with ELU42. FIG. 3 shows a direct comparison of the distance between opposing cartilage endpoints, as between 15-day treatment of biopsy punch wounds with GO-HA and 15-day treatment with ELU42.

### Example 3: Cartilage Regrowth in Rabbit Ears Following Administration of a Compound of the Present Disclosure

For the testing of compositions of the present disclosure for increased cartilage regeneration following wounding of the cartilage, in rabbits; an exemplary composition of the present disclosure was formulated as part of a phospholipid, as follows.

XAV939 (0.09 wt. %) was suspended in 2 mL (4.6 wt. %) of butylene glycol and 2 mL (4.6 wt. %) of polysorbate 20 and heated to 60° C. for 10 min while stirring. The mixture was then placed in a sonication bath and sonicated for 30 minutes. Phosphatidylcholine (1.2 wt %) was slowly dissolved in 2 mL (4.6 wt %) of ethanol. Once dissolved, the solution was added the XAV939/butylene glycol/polysorbate 20 mixture and stirred at room temperature for 5 min and then subjected to sonication for 30 minutes. After 30 minutes, the solution was added to a previously made solution of GO-HA (0.09 wt. %) in water (84%) and this new solution was sonicated for 30 min. After sonication, hydroxypropylcellulose (0.75 wt. %) was added to the mixture and the mixture stirred at room temperature for 24 hours before use, providing a liposomal formulation of ELU42.

New Zealand White rabbits were purchased from the Western Oregon Rabbit Company. Animals were anesthetized by way of either inhalation isoflurane (1-5% Iso/2.0 L O₂) or cocktail of Ketamine/Zylazine administered intramuscularly, for the least amount of time possible for completing the biopsy procedures described herein. Before surgery, the hair on the ventral side of each ear was removed thoroughly with a razor or other appropriate methods (e.g. depilatory) and biopsy sites surgically scrubbed with Clorhexidina and alcohol. Wounds of 6 mm size were created via punch biopsy using a disposable biopsy punch, with the wound extending throughout the entirety of the ear. Up to 8 total wounds per animal were created, and the excised tissue discarded. Following creation of wounds, the wound bed was cleaned with sterile saline and/or gauze to remove any foreign matter/loose tissue debris, if necessary.

Wounds were dosed with 0.3 mL of either saline or 0.3 mL of 1 mg/mL liposomal formulation of ELU42, every other day for 21 days. On each dosing day the wound surface was cleaned to remove any blood clots, excessive exudates, dressing debris, test article residues, or tissue build up on the wound bed, using sterile materials (i.e. saline moistened sterile gauze), but care was taken to avoid disturbing the surface of the wound. The test article was applied directly to the designated wound sites in a thin layer evenly over the wound site using a sterile applicator. After each dose the wound site was covered/bandaged in accordance with the study protocol or as recommended by the attending veterinarian. Images were taken on each day of dosing after the wounds were cleaned thoroughly and prior to dose administration on dosing days. Each image was taken directly in front of the wound to ensure an accurate measurement and included a ruler with appropriate identifiers.

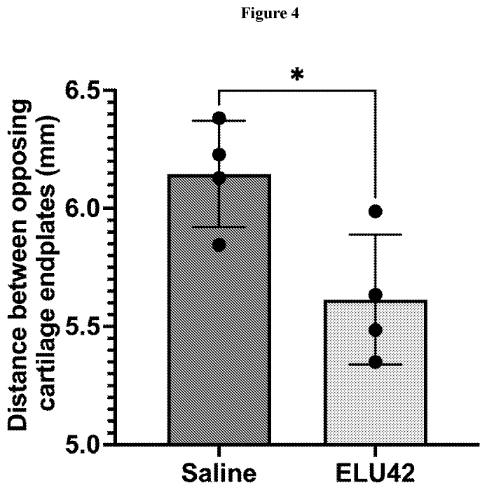

FIG. 4 shows a direct comparison of the distance between opposing cartilage endpoints, as between 21-day treatment of biopsy punch wounds through the rabbit ears as between with saline and treatment with liposomal formulation of ELU42. As can be seen, ELU42 has a statistically significant improvement (ANOVA *p<0.05, unpaired t-test) of the regeneration of cartilage, as compared to saline.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

1. A method for stimulating regeneration of mammalian cartilage subject to an injury comprising:
optionally identifying a mammal as experiencing an injury to cartilage;
optionally identifying the location and extent of the cartilage subject to an injury; and
contacting of said cartilage subject to an injury with an effective amount of a composition, the composition comprising
a matrix component comprising a graphene oxide (GO) and hyaluronic acid (HA) conjugate (GO-HA), wherein the GO and HA are covalently linked via a linker;
3,5,7,8-tetrahydro-2-[4-(trifluoromethyl)phenyl]-4H-thiopyrano[4,3-d]pyrimidin-4-one (XAV939); and
water;
optionally wherein XAV939 constitutes from about 0.001 wt % to about 5 wt % of the total composition.

2. The method of claim 1, wherein said cartilage subject to an injury is cartilage subject to an acute injury.

3. The method of claim 2, wherein said cartilage subject to injury is elastic cartilage.

4. The method of claim 1, wherein the composition further comprises a surfactant.

5. The method of claim 4, wherein the surfactant is polyethylene glycol (PEG), or a poloxamer.

6. (canceled)

7. The method of claim 5 wherein the PEG is in an amount of from about 0.1 wt % to about 20 wt % of the total composition.

8. The method of claim 1, wherein the composition further comprises a thickener.

9. The method of claim 8, wherein the thickener comprises hydroxypropyl cellulose (HPC).

10. (canceled)

11. The method of claim 1, wherein the linker comprises one or more —CH₂CH₂O— units.

12. The method of claim 1, wherein the linker comprises —Rₓ—R_{S}—R_{y}—, wherein Rₓ and R_{y} are each independently selected from the group consisting of —CO—, —COO—, —NH—, —NH—NH—, —NH—NH—CO—, —CS—, —S—, and —O—, and wherein R_{S} is an unsubstituted, saturated or unsaturated, linear alkylene group having 2-20 backbone carbons.

13. The method of claim 12, wherein Rₓ and R_{y} are each —NH—NH—CO—.

14. The method of claim 1, wherein the weight ratio of XAV939 to GO-HA is from about 1:2 to about 2:1.

15. (canceled)

16. The method of claim 1,
wherein the contacting is once every 48 hours.

17. The method of claim 16, wherein said cartilage subject to an injury is cartilage subject to an acute injury.

18. The method of claim 17, wherein said cartilage subject to injury is elastic cartilage.

19. The method of claim 16, wherein the composition further comprises a surfactant.

20-22. (canceled)

23. The method of claim 16, wherein the composition further comprises a thickener.

24-28. (canceled)

29. The method of claim 16, wherein the weight ratio of XAV939 to GO-HA is from about 1:2 to about 2:1.

30.-60. (canceled)

61. A pharmaceutical composition comprising 3,5,7,8-tetrahydro-2-[4-(trifluoromethyl)phenyl]-4H-thiopyrano[4,3-d]pyrimidin-4-one (XAV939), wherein the pharmaceutical composition is a liposomal composition and wherein the pharmaceutical composition comprises XAV939; a matrix component comprising a graphene oxide (GO) and hyaluronic acid (HA) conjugate (GO-HA), wherein the GO and HA are covalently linked via a linker; water; and a phospholipid.

62. The pharmaceutical composition of claim 61, wherein the phospholipid is phosphatidylcholine.

63. The pharmaceutical composition of claim 61, wherein the pharmaceutical composition further comprises a butylene glycol.

64. The pharmaceutical composition of claim 61, wherein the pharmaceutical composition further comprises a polysorbate 20.

65. The pharmaceutical composition of claim 61, wherein the pharmaceutical composition comprises butylene glycol, a polysorbate 20, phosphatidylcholine, ethanol, GO-HA, water, and hydroxypropylcellulose.

66. The pharmaceutical composition of claim 61, wherein the pharmaceutical composition further comprises another pharmaceutical or therapeutic compound selected from the group consisting of an antifibrotic compound, an anti-cancer agent, an anti-inflammatory agent, an analgesic, an antibiotic, a corticosteroid, a cytotoxic drug, an antiseptic, nicotine, an anti-platelet drug, an NSAID, colchicine, an anti-coagulant, a vasoconstricting drug, an immunosuppressive, a growth factor, an antibody, a protease, a protease inhibitor, an antibacterial peptide, an adhesive peptide, a hemostatic agent, living cells, honey, and nitric oxide.

67. A method for stimulating regeneration of mammalian cartilage subject to an injury comprising:
optionally identifying a mammal as experiencing an injury to cartilage;
optionally identifying the location and extent of the cartilage subject to an injury; and
contacting of said cartilage subject to an injury with an effective amount of a composition of claim 61.
